(19) United States
(12) Patent Application Publication
(10) Pub. No.: US 2005/0261633 A1
(43) Pub. Date: Nov. 24, 2005

(54) Rechargeable handheld injection device with reversible drive having adjustable syringe cradle

(76) Inventor: Ben M. Khalaj, San Clemente, CA (US)

Correspondence Address: Ben M. Khalaj, 106 Arista, San Clemente, CA, 92673, US

(21) Appl. No.: 11/129,972

(22) Filed: May 16, 2005

Related U.S. Application Data
(60) Provisional application No. 60/572,090, filed on May 19, 2004.

(51) Int. Cl. A61M 5/00

(52) U.S. Cl. 604/181; 604/227

(57) ABSTRACT

It is an object of the present invention to provide a rechargeable handheld injection device that supports stock syringes for purposes of injection/removal of fluid or fat cells. This injection device comprising a housing, an adjustable/replaceable syringe cradle assembly, an electrical actuator with settings of dual speed that actuate the syringe cradle assembly in forward and reversible drive using forward and reverse buttons, a trigger switch to control the actuator, a pre-programmed syringe control panel, a needle guard assembly and a rechargeable battery in conjunction with ON/OFF switch button and electronic component. A separate wall mount battery charger is used to charge the battery of this handheld device.

Applicant claim priority-filling date for this application. A Provisional application has been filed on May 19, 2004, and the application No. is 60/572,090.

### FIELD OF THE INVENTION

The present invention relates to an injection device to be used with standard stock syringes. More particularly, the invention relates to an injection device which includes injection mode to inject fluid in forward mode and having reverse mode to withdraw fat cells in reversible mode.

### BACKGROUND OF THE INVENTION

For many years doctors have been using injection devices for various medical purposes including but not limited to mesotherapy, hydropuncture, vaccinations, and body sculpting. Injection of fat into known as Autologous or micro-lipoinjection procedures involves using a patient's own fat to enhance features, improve contour, and correct defects through sculpting the body. To withdraw fat from a patient's body, fat is in general taken from the thigh or abdominal area and a fat removal device is used. Once the fat is removed, it is processed to remove excess fluids, and finally the fat is inserted beneath the skin for means of body sculpting. A handheld injection device with a reversing drive would be greatly beneficial to doctors performing fat transfer on their patients.

Some prior patents refer to other handheld pistols. For example, U.S. Pat. No. 5,034,003, issued to Raymond Denance, disclosed “An injection device comprising a support stock that supports a syringe having a plunger and a needle. The plunger is movable in an axial direction to force liquid from the syringe through the needle. A safety valve prevents flow of liquid from the syringe to the needle. A stabilizing sight is provided, for application against skin during an injection. The stabilizing sight is mounted on the device for movement relative to the device such when the device is pressed against the skin the needle moves from a retracted position relative to the stabilizing sight to an advanced position relative to the stabilizing sight to penetrate the skin.”

Another U.S. Pat. No. 4,108,177, issued to Michel Louis Paul Pistor, disclosed ” An automatic injector device operating stroke-by-stroke, comprising a frame whose shape is substantially similar to that of a revolver, a removable injection syringes comprising a syringe body, a syringe piston and at least one injection needle, a syringe-cradle carrying the said syringe and itself supported by the said frame, drive means on the one hand actuating in reciprocating motion the cradle-syringe assembly, and on the other hand actuating the syringe piston, and means for manually starting at least one injection, such as for example a trigger, which operates at least a part of said drive means.”

In spite of the advantages of the devices in prior art, there are some limitations which cannot be delivered for the purposes of extracting or removing fat cells for fat transfer procedures, with respect to the reversible function mode and its reverse drive. It would be beneficial to have a forward mode and a reverse mode in one injection device, which has a set of pre-programmed functional tasks as its necessary for medical procedures.

This invention overcomes the shortcomings of conventional devices by providing a rechargeable handheld injection device which can accommodate a variety of stock syringes and can inject or withdraw fluid/fat cells into/from the body for the propose of medical treatments.

### SUMMARY OF THE INVENTION

It is an object of the present invention to provide a rechargeable handheld injection device that uses stock syringes for the purposes of injection/removal of fluid or fat cells. This injection device comprising a housing, an adjustable syringe cradle assembly, an electrical actuator with settings of dual speed that actuate the syringe cradle assembly in forward and reversible drive using forward and reverse buttons, a trigger switch to control the actuator, a control panel with a set of pre-selected program and function keys, a needle guard assembly and a rechargeable battery. A separate wall mount battery charger is used to charge the battery of this handheld device. The actuator is an electric stepper motor which drives syringe cradle assembly via meshing gears is programmed for continuous or simultaneous forward and reverse mode via the forward and reverse buttons.

It is another object of this invention to place a variety of syringe sizes into syringe cradle assembly. Cradle assembly can accommodate syringes for 1, 3, 5, and 10 ml/cc. Furthermore, when loading the syringe's plunger into the cradle assembly some adjustment may require with respect to size and the length of the syringe.

There are two different settings for this adjustment. Once the locking/unlocking lid of cradle is open, the cradle can be released and move freely in coaxial with syringe and also can be adjusted in up and down which is in Y-axis.

It is yet another object of this invention to provide two types of syringe cradles. One is a long syringe cradle for syringes with long needles and the other is a short syringe cradle for syringes with short needles. It is depending on the length of the syringe needle. The syringe cradle should be exchanged so that the needle does not protrude beyond the needle guard. In addition, the depth that the needle will penetrate the skin can be adjusted using the depth-adjusting knob. This adjustment affects how far the needle guard will travel into the housing once activation occurs and thus how deep the needle will penetrate the skin, it will not result in a shift of the needle guard until activation occurs. This depth adjustment can be viewed via a shift of the red indicator located in a transparent window.

Further objects and advantages of this invention will become apparent from consideration of the drawings and descriptions that follow.

### BRIEF DESCRIPTION OF THE DRAWINGS

Exemplary embodiments of the invention will now be described in conjunction with the drawings in which:

FIG. 1 is a perspective view of the present invention showing an injection device with the syringe cradle lid in the unlocked position for forward mode drive. The wall mount battery charger showing away from the device.

FIG. 2 is a perspective view of the present invention showing an injection device with the syringe cradle lid in the locked position for reverse mode drive.

FIG. 3 is an exploded view of the injection device shown in FIG. 1-2.

FIG. 4 is an exploded view of the injection device with half of the housing removed and the needle guard assembly is shown in cross section.

FIG. 5 is a partial view taken along line 5-5 in FIG. 4 showing the syringe selection panel.

FIG. 6 is a partial cross section of the side view of the injection device.

FIG. 7 is a side perspective view of the injection device shown in forward motion.

FIG. 8 is a side perspective view of the injection device shown in reverse motion.

FIG. 9 is a perspective view of the adjustable syringe cradle assembly.

FIG. 10 is a top view of the adjustable syringe cradle assembly.

FIG. 11 is a side view of the adjustable syringe cradle assembly.

FIG. 12 is an exploded view of the adjustable syringe cradle assembly.

### DETAILED DESCRIPTION OF THE INVENTION

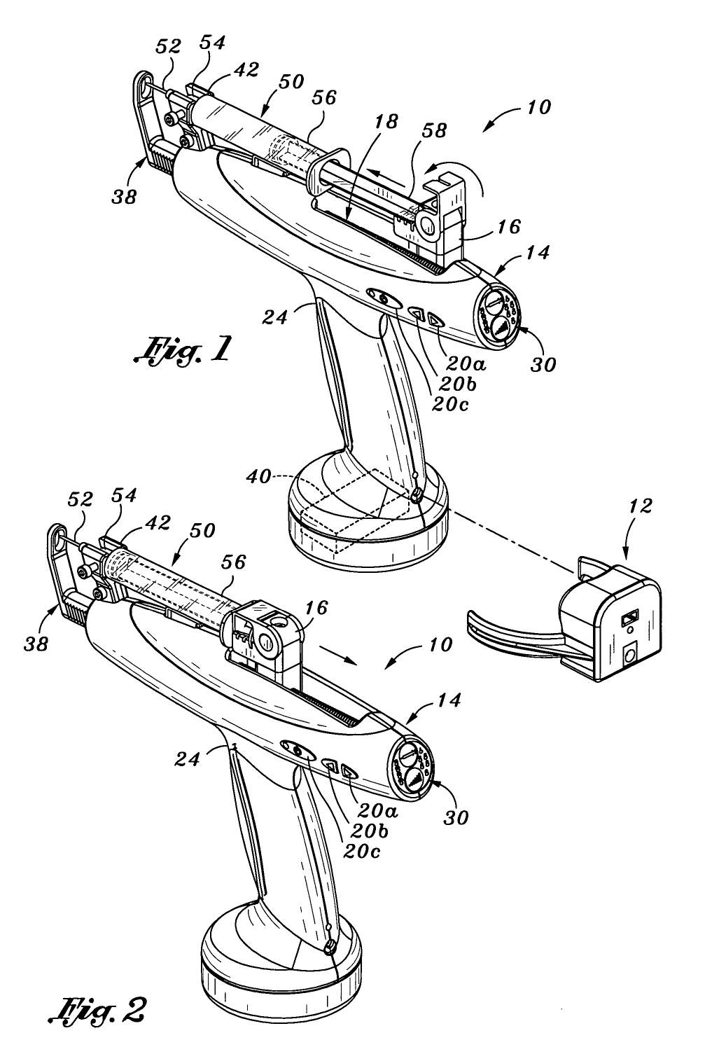

FIG. 1 is a perspective view of the present invention shown a handheld injection device 10 with a wall mount battery charger unit 12 away from it. This injection device 10 as seen in FIGS. 1 & 2, comprising a housing 14, an adjustable syringe cradle assembly 16, an electrical actuator 18 with settings of dual speed that actuate the syringe cradle assembly 16 in forward and reversible drive using forward and reverse buttons 20a-20b, a trigger switch 24 to control the actuator, a control panel 30 with a set of pre-programmed function keys 32, a needle guard assembly 38 and a rechargeable battery 40 in conjunction with ON/OFF switch button 20c. A separate wall mount battery charger 12 is used to charge the battery 38 of this handheld device 10.

A stock syringe 50 normally comprising a needle 52, a neck 54, body 56 and a plunger 56.

In order to load a stock syringe 50 onto the handheld injection device 10, a fixed frontal clamp 42 which is shaped like a “Y” is located on the handheld housing 14. This frontal clamp 42 secure the syringe neck 54 and, on the other end, the syringe plunger can be placed into a movable/adjustable cradle assembly 16 which actuate by driving means of the electrical/mechanical components of the device in forward or reverse mode.

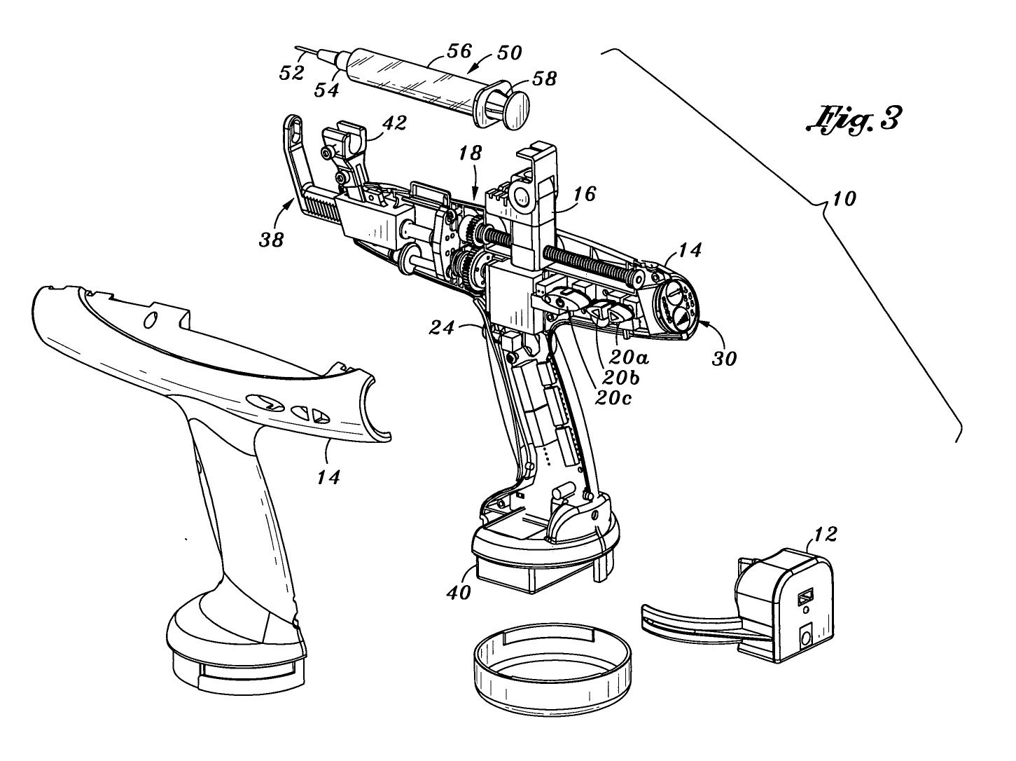

FIG. 1 shows the syringe is loaded position but the cradle locking/unlocking lid 64 is still open to make necessary adjustment before deploying and driving the plunger for injection means. Nevertheless, FIG. 2 shows the loaded syringe is ready for reversal mode and cradle assembly is pulling the syringe plunger in axial direction to remove fat cells from the body.

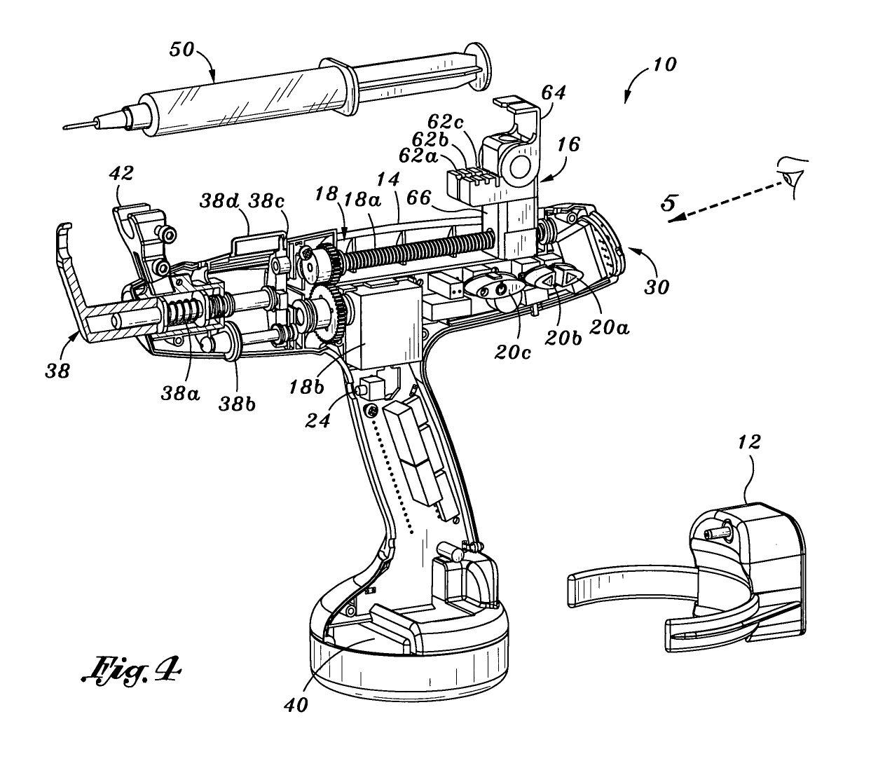

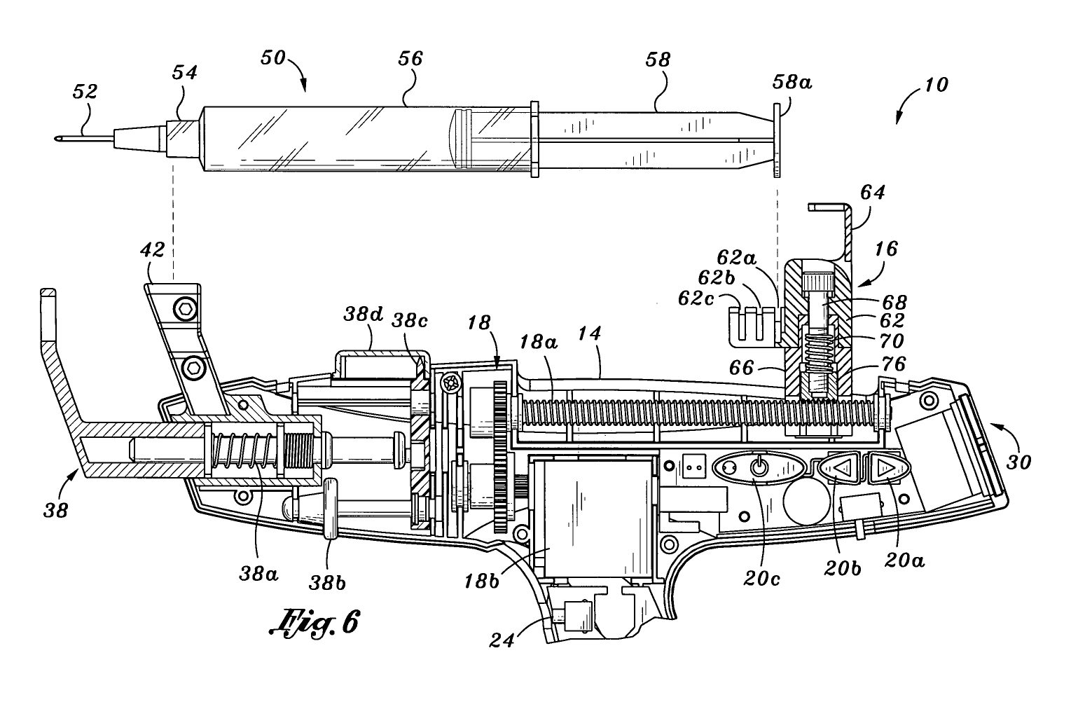

Now, referring to FIG. 3-4, the handheld device 10 is shown in more details by removing the half of the housing and expanding the parts to the side. An adjustable needle guard assembly 38 is located at the front end of the handheld device 10. The adjustable needle guard 38 is placed to protect the syringe needle from accidents and provide safety. This needle guard 38 can be adjusted with respect to needle position when the needle is inserted into the tissue and provide desired depth of needle to penetrate the tissue when injecting or when removing fat cells. The Adjustable needle guard 38 is retractable via a loaded spring 38a and a depth adjusting knob 38b also provide how far the needle guard 38 needs to retract back and fort with respect to syringe needle 52 position as best seen in FIG. 6. Furthermore, a depth indicator 38c shows what the depth would be with respect to needle guard position and can be viewed via a transparent window 34d on the top of the handheld housing 14.

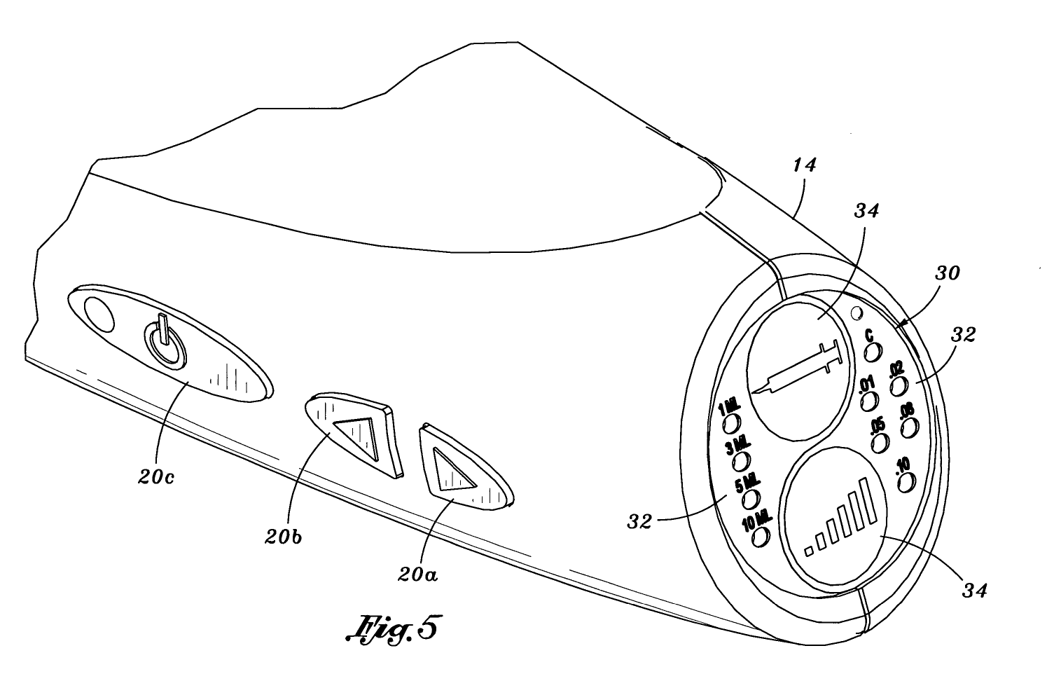

The control switch panel 30 of the handheld injection device 10 is shown in FIG. 5. The panel 30 has a set of pre-selected program to activate a predetermined task for any stock syringes and the graphical display 34 indicates the progress of the selected mode either in forward mode or in reversal mode. The pre-selected programs can be for continuous or simultaneous via the forward and reverse buttons as the trigger switch 24 is activated. Another advantage of this control switch panel 30, is to make sure the progress of injected medication is fully deployed. The graphics on control panel shows such a progress and when the medication is fully injected, the control panel is notifying the user by a beep sound and a blinking led. The specific timing of each dose is calculated with respect to the injection process of each stock syringes. The benefit of such notification is to inject the medication fully into the tissue and not to waist it especially when the medication is expensive.

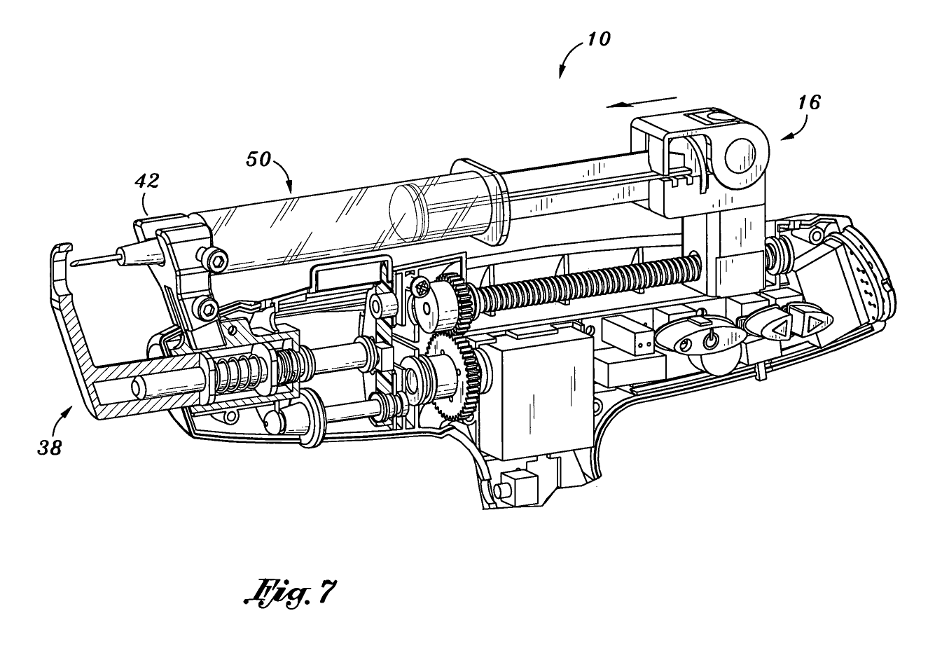

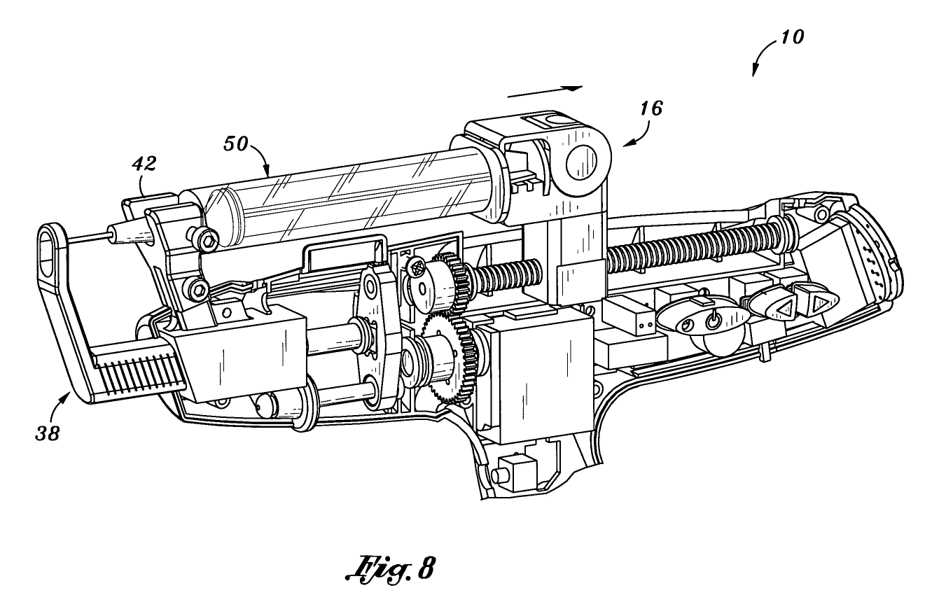

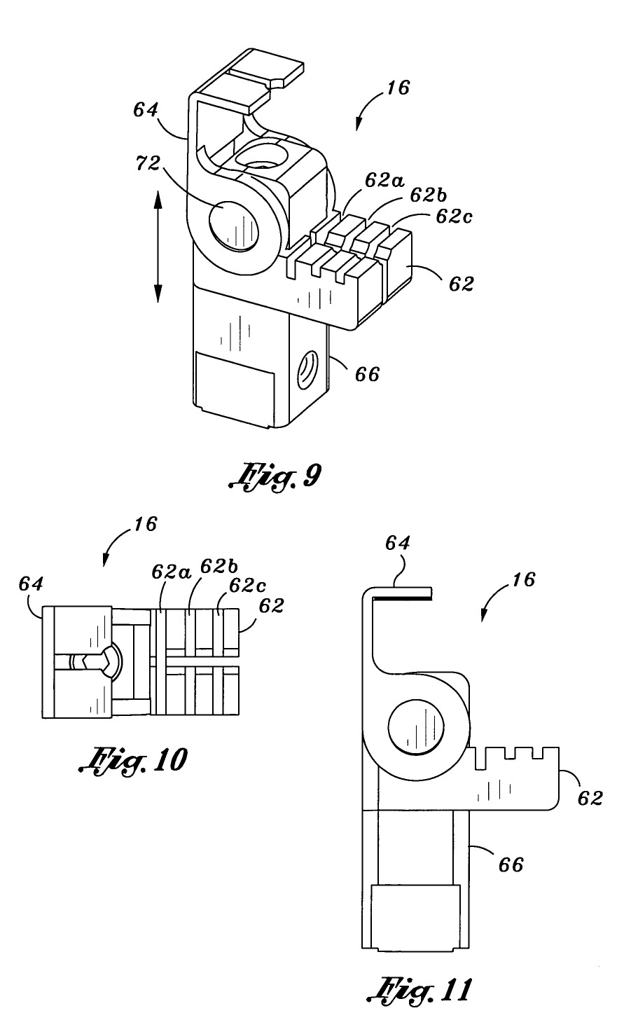

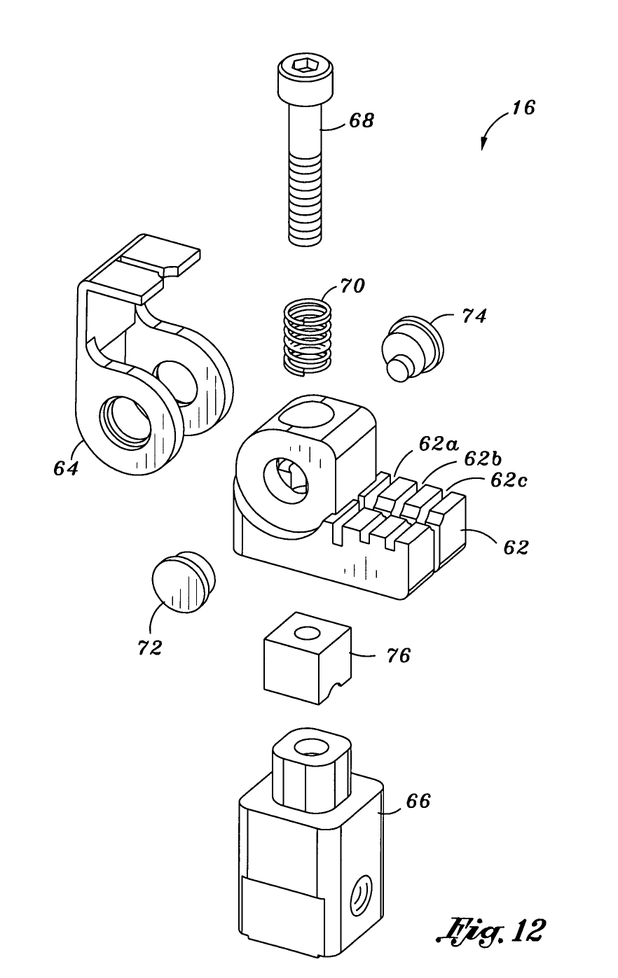

Referring to FIG. 6-12, the Adjustable syringe cradle assembly 16 is driven by warm gear 18a that actuate back and fort in coaxial direction. The actuator is an electric stepper motor 18b which drives syringe cradle 16 by selecting per-selected program on the control panel and the function keys. Cradle assembly 16 can accommodate syringes 50 for 1, 3, 5, and 10 ml/cc. Cradle assembly 16 comprising a main body 62 that has pluralities of slots 62a, 62b, 62c to accommodate stock syringes 50 via its plunger's end 58a, a locking/unlocking lid 64, and a movable meshing gear block 66 that is driven by electrical/mechanical actuator 18. Cradle assembly 16 can be adjusted in X-axis and Y-axis when the cradle lid is opened and by loading the syringe plunger's end 58a into the cradle slots 62a, 62b, 62c and locking the cradle lid by closing it to secure the syringe plunger into the cradle assembly. When the cradle lid is opened, some adjustment may require with respect to size and the length of the syringe. There are two different settings for this adjustment. Once the locking/unlocking lid of cradle is open, the cradle meshing gear block 66 can be released and freely move in coaxial with syringe 50 and also it is self adjusted in up and down which is in Y-axis by the a spring 68. The other advantageous of this invention is to remove and replace the cradle assembly for oversize syringes. There are two types of syringe cradles provided in this invention. One is for a long syringe with long needles and the other one is for a short syringe with short needles. It is depending on the length of the syringe needle. These types of syringe cradles should be exchanged so that the needle does not protrude beyond the needle guard. Another advantage of this removable syringe cradle 16 is for sterilization purposes.

While this invention is susceptible of embodiments in many different forms, this specification and the accompanying drawings disclose only some specific forms as examples of the invention. The invention is not intended to be limited to the embodiments so described; however, the scope of the invention is pointed out in the appended claims.

1. A handheld injection device to be used in conjunction with stock syringes having a needle, a body, and a plunger comprising:
a housing; said housing accommodates mechanical and electrical components,
an adjustable syringe cradle assembly, said syringe cradle assembly supports stock syringe via said syringe plunger's end;
a frontal clamp, said clamp holds said syringe at its neck securely, said frontal clamp is positioned on the said housing near its front end;
drive means for actuating said syringe cradle assembly to move said syringe plunger in axial direction either in forward or reverse mode with respect to said syringe body;
a trigger switch, said trigger switch activates said drive means;
a needle guard assembly, said needle guard is placed at frontal end of said housing and retracts in axial direction with respect to injection operation;
a control switch panel, said switch panel is located at the rear end of said housing and can select specific tasks of said syringe and its dosages via pre-selected programs, and;
a rechargeable battery, said battery is the source of electricity for said electrical components.

2. A handheld injection device according to claim 1, wherein said adjustable syringe cradle assembly accommodates different sizes of stock syringes.

3. A handheld injection device according to claim 1 & 2, wherein said adjustable syringe cradle assembly has different adjustment settings to provide accommodations of said stock syringes with respect to said syringe's length and said syringe overall size.

4. A handheld injection device according to claim 3, wherein said adjustable syringe cradle assembly has different adjustment settings, furthermore the said setting can be in coaxial direction with respect to said syringe's length.

5. A handheld injection device according to claim 3, wherein said syringe cradle assembly has different adjustment settings, furthermore the said setting can be in direction of up and down adjustments with respect to said syringe overall size.

6. A handheld injection device according to claim 1, wherein said syringe cradle assembly is removable and can be replaced.

7. A handheld injection device according to claim 1 & 6, wherein said syringe cradle assembly can be in different size portion.

8. A handheld injection device according to claim 1, wherein said drive means has a dual speed.

9. A handheld injection device according to claim 1, wherein said drive means for actuating said syringe cradle assembly can be continuous drive.

10. A handheld injection device according to claim 1, wherein said drive means for actuating said syringe cradle assembly can be simultaneous drive.

11. A handheld injection device according to claim 1, wherein said needle guard assembly has an adjustable knob to displace said needle guard in axial direction with respect to said syringe needle's length.

12. A handheld injection device according to claim 1, wherein said a pre-programmed control panel, said panel includes graphical interfaces for ease said syringe tasks.

13. A handheld injection device according to claim 1, wherein said a pre-programmed control panel includes variety of tasks for said syringe for injection operation.

14. A handheld injection device according to claim 1, wherein said a rechargeable battery has a remote charger.

15. A handheld injection device according to claim 12, wherein said a remote charger can be a wall mount charger.
